(19) United States
(12) Patent Application Publication
(10) Pub. No.: US 2008/0281362 A1
(43) Pub. Date: Nov. 13, 2008

(54) DEVICE AND SYSTEM FOR CRANIAL SUPPORT

(76) Inventor: Jeremy Lemoine, Austin, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/746,481

(22) Filed: May 9, 2007

(51) Int. Cl. A61B 17/70

(52) U.S. Cl. 606/261; 606/246

(57) ABSTRACT

A device and system for supporting the cranium, and a method for manufacturing. Embodiments of the present invention provide fixed or rotatable angled connectors to connect the cranial support rods and support greater mechanical loads and stresses over sharper angles. Additionally, embodiments of the present invention include pre-shaped cranial support rods to reduce stress.

### FIELD OF THE INVENTION

This invention relates generally to supporting bony tissues within a body, and in particular to devices and systems for supporting the cranium.

### BACKGROUND OF THE INVENTION

In general, rods used for spinal stabilization are substantially straight with some curvature. These rods can be connected together using existing in-line connectors to lengthen the area of stabilization, In-line connectors can also be used to transition between support rods of various diameters. In some situations, however, rods must transition across an angle. For example, supporting the cranium may be desirable or even necessary. In prior art solutions, a support rod is generally bent by hand into a configuration by the doctor during the surgery, and then attached to the occipital bone and the cervical spine. However, bending the rod to contour to a curvature may be difficult due to the mechanical properties of the rod, the complexity of the curvature, the strength of the doctor, and the time constraints of surgery.

A disadvantage with prior art support rods is the undesirable mechanical properties that result from working the rod. Manipulating the rod, such as bending or twisting, is undesirable because the decrease in mechanical properties generally weakens the rod, and the possible introduction of cracks that can lead to failure. Those skilled in the art will appreciate that these cracks may be microscopic, and the amount the rod is weakened will not be known to the doctor, so the doctor will not know if the device can provide the necessary support. Additionally, manipulating a rod increases the time needed for a medical professional to prepare the support rod for implantation.

One prior art approach to reducing cracking in a rod has been to increase the diameter of the rod. Although a larger rod can generally support more weight, the rod also requires more work to configure it to contour to the curvature of the spine, and may still develop sufficient cracks that may lead to failure, or may have generally reduced strength, Furthermore, the larger diameter rods will also be less likely to allow movement of any amount in any plane.

A related approach to using larger rods is to utilize a rod of varying diameter, such that the diameter of the rod is larger for bridging sharp angles and thinner when the rod is generally straight. These rods are disadvantageous for the same reasons listed for larger diameter rods in general, plus the possibility that the rod is bent incorrectly such that the stresses on the rod are not carried by the thickest portion of the rod. It is therefore desirable to provide a device and system that does not rely on the skill of a medical professional to configure the device to distribute forces.

Another disadvantage with prior art methods of bending rods, particularly plate-less cranial support rods that must be bent in surgery, is the resulting minimal contact with the occipital bone and increase in stresses. In situations in which embodiments of the present invention are used without an occipital support plate (i.e. plate-less systems) the weight of the cranium supported by the cranial support rod is distributed over the small surface area of the rod, resulting in large stresses Large stresses may cause pain, sores, fractures, or other undesirable side effects in a patient, and may also damage the cranial support rod. It is therefore desirable to provide a way to maximize the area in contact with the bone such that undesirable side effects are minimized or avoided. It is further desirable to provide a rod that may safely cradle the occipital bone to prevent damage to either the occipital bone or the rod.

### SUMMARY OF THE INVENTION

Embodiments of the present invention are advantageous over prior art stabilization systems for supporting the cranium. For example, embodiments of the present invention allow medical professionals to support the cranium using a thin cranial support rod that is pre-formed to contour the curvature of the spine. In these embodiments, the present invention is advantageous over prior art methods by reducing the size of the support rod, yet maintaining the strength and other desired mechanical properties. In situations in which the medical professional must still bend or modify the present invention, embodiments of the present invention are advantageous because the internal stresses have been reduced or eliminated by heat treating the support rod. As a result, the support rod is able to support the cranium at sharper angles than cranial support rods that have not been pre-configured and/or heat-treated. Furthermore, embodiments of the present invention enable the cranial support rod to be used with a connector that can be configured to a selected angle such that the angled connector supports the loads across sharp angles.

Embodiments of the present invention provide devices, systems and methods for supporting the cranium and stabilizing movement across the occipital-cervical junction. One embodiment of the present invention includes a cranial support rod with a first section shaped to contour the curvature of a first portion of a body, and a second section configured to contour a second portion of the skeleton, wherein the rod has a bent shape selected to bridge the occipital-cervical junction and is heat-treated. The portions of the spine may include the cervical portion of the spine and the occipital-cervical junction. In this embodiment, the cranial support rod may be configured by casting, forging or extruding a rod, either directly into a support configuration or into an initial configuration and then bending, twisting, or otherwise working into a support configuration. Once the rod is configured to contour to a curvature of a portion of the body, the rod is annealed or otherwise heat treated to remove internal stresses created during the process of configuring the rod into the support configuration. The cranial support rod may be manufactured from a biocompatible material such as stainless steel or titanium, or any other material that can be configured and then heat-treated to reduce stress in the cranial support rod.

Another embodiment of the present invention includes an angled connector having two sections that are configurable to maintain two support rods in a selected configuration. The angled connector is adapted to securely receive and maintain the support rods at a selected angle. In some embodiments, the angled connector is fixed at a pre-selected angle such as thirty, forty-five, ninety, one-hundred thirty five, or one-hundred eighty degrees. In some embodiments in which the two sections are rotatably connected, the angled connector is configurable to one of the pre-selected angles, or the angled connector may be configurable to any angle within a given range, such as between thirty and one-hundred eighty degrees.

Yet another embodiment of the present invention is directed to a system for supporting the cranium, an angled connector comprising a first receiver adapted for secure connection to a first support rod extending in a first direction from the first receiver, and a second receiver connected to the first receiver and further adapted for secure connection to a second support rod extending in a second direction from the second receiver, wherein the first receiver and second receiver are connected to maintain an angle between the first and second directions, a first support rod connected to the first receiver, and a second support rod connected to the second receiver The angled connector may he pre-configured, such as by machining or casting, to have a selected angle, or may be configurable during a surgical procedure by a medical professional to have a desired angle.

Yet another embodiment of the present invention is directed to a method of supporting the cranium by attaching a first section of a cranial support rod to a first portion of the body and a second section of the cranial support rod to a second portion of the body, wherein the cranial support rod comprises a first section shaped to contour the curvature of the first portion of a body, and a second section and wherein the rod is in a configuration selected to bridge the occipital-cervical junction and is heat-treated. In some embodiments, the rod is shaped to contour to an occipital bone or to contour to a portion of the cervical spine.

Yet another embodiment of the present invention is directed to a method for supporting the cranium by connecting a first support rod to an angled connector, wherein the angled connector comprises a first receiver adapted for secure connection to a first support rod extending in a first direction from the first receiver and a second receiver connected to the first receiver and further adapted for secure connection to a second support rod extending in a second direction from the second receiver, wherein the first receiver and second receiver are connected to maintain an angle between the first and second directions. A second support rod is connected to the angled connector, the first support rod is connected to a first portion of the body, and the second support rod is connected to a second portion of the body. In some embodiments the first support rod is connected to a portion of the cervical spine or an occipital bone. In some embodiments, the angled connector comprises a first receiver adapted for secure connection to the first support rod extending in a first direction from the first receiver and a second receiver connected to the first receiver and further adapted for secure connection to the second support rod extending in a second direction from the second receiver, wherein the first receiver and second receiver are connected to maintain an angle between the first and second directions. In some embodiments the angle is between thirty and one-hundred eighty degrees. In some embodiments the connection between the first and second receivers comprises a rotatable connection. In some embodiments the rotatable connection is configurable to pre-set angles such as such as thirty, forty-five, ninety, one-hundred thirty five, or one-hundred eighty degrees.

Embodiments of the present invention overcome prior art devices and systems for supporting the cranium by utilizing a cranial support rod of minimal thickness while maintaining desirable mechanical properties.

The present invention overcomes prior art devices and systems by providing an angled connector in conjunction with cranial support rods to support the weight of the cranium and bridge the occipital-cervical junction.

These, and other, aspects of the invention will be better appreciated and understood when considered in conjunction with the following description and the accompanying drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

### BRIEF DESCRIPTION OF THE DRAWINGS

A more complete understanding of the present invention and the advantages thereof may be acquired by referring to the following description, taken in conjunction with the accompanying drawings in which like reference numbers indicate like features and wherein:

FIGS. 1A and 1B are side views of one embodiment of a cranial support rod;

FIG. 2A is a side view of one embodiment of an angled connector;

FIG. 2B is an isometric view of one embodiment an angled connector;

FIGS. 3A, 3C, and 3D are side views of one embodiment of an adjustable angled connector;

FIG. 3B is an isometric view of one embodiment of an adjustable angled connector; and

FIG. 4 is a side view of one embodiment of a cranial support system

### DETAILED DESCRIPTION

The invention and the various features and advantageous details thereof are explained more fully with reference to the non-limiting embodiments that are illustrated in the accompanying drawings and detailed in the following description Descriptions of well known starting materials, processing techniques, components and equipment are omitted so as not to unnecessarily obscure the invention in detail. Skilled artisans should understand, however, that the detailed description and the specific examples, while disclosing preferred embodiments of the invention, are given by way of illustration only and not by way of limitation. Various substitutions, modifications, additions or rearrangements within the scope of the underlying inventive concept(s) will become apparent to those skilled in the art after reading this disclosure.

Reference is now made in detail to the exemplary embodiments of the invention, examples of which are illustrated in the accompanying drawings. Wherever possible, the same reference numbers will be used throughout the drawings to refer to the same or like parts (elements).

The systems and methods of the invention may be particularly useful for supporting or and stabilizing movement by bridging the occipital-cervical junction and thus it is in this context that embodiments of the invention may be described.

It will be appreciated, however, that embodiments of the systems and methods of the present invention may he applicable for supporting bony tissue or stabilizing movement across junctions in other areas of the spine or body. For purposes of this document, the terms stabilize and stabilization refer to the control of one or more degrees of freedom for movement. Stabilization may result in the complete restriction of movement about a particular axis or plane, or it may limit the movement over a selected range of motion.

Prior art cranial support rods rely on the medical professional to skillfully bend, twist and otherwise work the cranial support rod into the proper configuration. The varying knowledge, skill, and even strength of the medical professional could affect the amount of bending necessary to configure the cranial support rod, resulting in a wide variance in the strength and mechanical properties of the cranial support rod. In contrast, the present invention minimizes reliance on the knowledge, skill, or strength of the medical professional to the same degree, to properly manipulate the cranial support rod. The variance in strength and mechanical properties is therefore minimized.

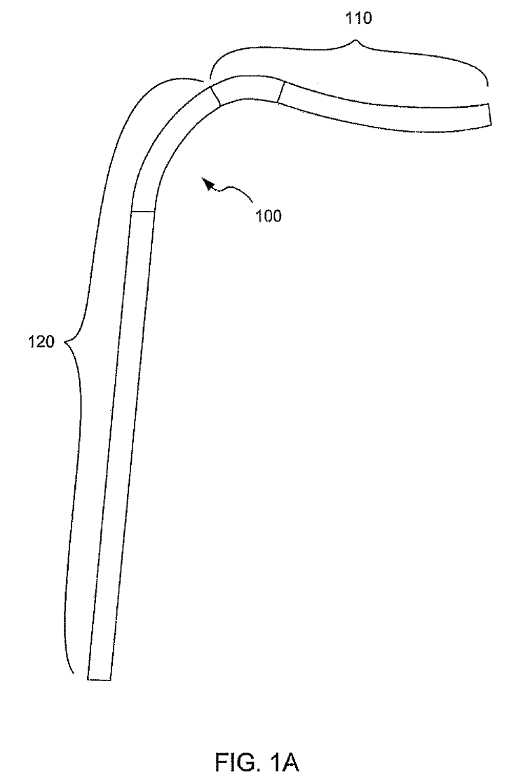

Embodiments of the present invention may be better explained with reference to FIGS. 1A and 1B which depict side views of a cranial support rod 100 in accordance with one embodiment of the present invention. In FIGS. 1A and 1B, cranial support rod 100 generally describes a rod having essentially the same dimensions, such as cross-sectional profile and diameter, throughout its length, and divided into two or more curved sections 110 and 120, with each section 110 and 120 pre-configured to contour curvatures of the body. For purposes of this document, the cross-sectional dimensions of rod 100 are generally referred to as diameters. However, the present invention is not so limited, and various cross-sectional profiles may be utilized without departing from the scope of the invention. Square, hexagonal, and oval are examples of other cross-sectional profiles possible with the present invention.

Cranial support rod 100 may be manufactured from any material having mechanical properties necessary to stabilize movement of the cranium (e.g. support weight) and may have additional mechanical properties desirable for other functionality, such as flexibility or torsional stiffness. In preferred embodiments, the cranial support rod 100 is manufactured from biocompatible material, such as titanium, stainless steel, or cobalt chromium. In preferred embodiments, cranial support rod 100 is manufactured from commercially pure Grade 4 titanium.

In some embodiments, cranial support rod 100 is cast, extruded, forged or otherwise configured to contour selected curvatures of the spine or the profile of the occipital bone. In some embodiments, cranial support rod 100 is cast, extruded, or otherwise formed to an initial form and then bent, rolled, twisted, forged or otherwise configured to contour to the curvature of part of the spine or the occipital bone. Advantageously, pre-configuring cranial support rod 100 to contour a selected curvature of the body enables a larger portion of cranial support rod 100 to contact the selected part of the body, cradling the cranium, thereby reducing point stresses, pain, sores and other undesirable characteristics of prior art devices.

After cranial support rod 100 is in a selected configuration, cranial support rod 100 is stress-relieved, such as by forging or heat-treating, to decrease or eliminate the internal stresses created when cranial support rod 100 was manufactured into the configuration. In this respect, the present invention is advantageous over prior art support rods because the smaller diameter cranial support rod 100 is now able to provide more stabilization and control (e.g. support more weight) than possible without the heat treatment process, and possibly provide more stabilization control than some larger diameter rods that have internal stresses and cracks. Furthermore, in situations in which a smaller diameter cranial support rod 100 has been pre-formed to accommodate large bends but still needs some manipulation during implantation, the origination and propagation of cracks is reduced and the change in mechanical properties is minimized.

Heat-treating, particularly annealing, reduces internal stresses and cracks that are the result of working the metal, such as by rolling, coiling, bending, compressing, torquing, or pulling the workpiece, Heat-treating even a small amount may result in increased fatigue strength, yield strength, tensile strength, or other mechanical properties. Annealing as a form of heat treating is effective for reducing the stresses in rod 100 and for reducing the number and size of cracks in rod 100. Annealing generally involves heating the material to a desired temperature (usually above 1040 degrees Celsius), holding the material at the desired temperature for a selected period of time, and then cooling the material at a rate selected based on desired properties. Those skilled in the art will appreciate that the temperature and time for a particular heat-treatment process may be selected based on the material, desired mechanical properties, processes used to work the material, or any combination without departing from the scope of the present invention. Quench annealing, stabilizing annealing, and process annealing are examples showing the variation in annealing processes.

An advantage to pre-forming or configuring the cranial support rod 100 and stress-relieving (egg. forging or annealing) it to a predetermined stress state is the control in variance between rods 100. In other words, a doctor using a cranial support rod 100 of the present invention will have a high degree of certainty that the rod 100 can support the weight for which it is intended. Unlike prior art rods that were weakened during the configuration process, cranial support rods 100 of the present invention may be tested after the rod 100 has been stress-relieved to determine the mechanical properties. For example, a random sample from a batch of cranial support rods may be tested to predict the strength or other mechanical property of a cranial support rod 100 from the batch. Knowing the mechanical properties of the rod 100, a doctor may use a particular cranial support rod 100 based on a desired characteristic or functionality. A pre-bent rod without the appropriate stress relief would be able to withstand less bending, both in terms of the number of bends and the degree of bending. Through annealing and/or stress relieving after the pre-bending, the surgeon now has more ability to bend a greater number of times without risking rod breakage. Advantageously, rod 100 is already shaped to contour to the occipital bone and the cervical spine, and configured at an angle to bridge the occipital-cervical junction, so fewer bends (if any) are needed. Additionally, heat treating may provide more corrosion resistance, which is particularly desirable for devices implanted in the body.

In some embodiments, rod 100 may further include a layer (not shown) of material selected for biocompatibility purposes such as infection resistance, improved visibility during implantation or during a fluoroscopy, or improved mechanical properties such as corrosion resistance.

In the embodiments shown in FIGS. 1A and 1B, cranial support rod 100 is divided into two sections, 110 and 120. First section 110 and second section 120 are each shaped to contour to a selected portion of the body and rod 100 further defines an angle between sections 110 and 120.

In various embodiments, sections 110 and 120 may be of different lengths, have different cross-sectional dimensions, and each section 110 and 120 may have a different shape, such as radius of curvature. Cranial support rod 100 is not limited to two sections, but may have more sections, with each section having a selected length, diameter, and radius of curvature to more closely contour a selected portion of the body. For example, in FIG. 1B, one embodiment of a cranial support rod 100 is shown as it relates to the cervical portion of the spine and the occipital bone. Section 110 has a length and radius of curvature configured to contour to the occipital bone, and section 120 has a length and radius of curvature 120 configured to contour to the cervical spine. Those skilled in the art will appreciate that a section may be curved over its length, such as section 110, or may be curved over a portion of its length and have a straight portion, such as section 120.

Unlike other junctions in the spine (cervical-thoracic, thoracic-lumbar, lumbar-sacral), the occipital-cervical junction is defined as a sharp angle which requires an extensive bend or curvature in any support rod. To stabilize movement of the cranium across this junction, first section 110 and second section 120 extend in different directions generally defined by the angle corresponding to the occipital-cervical junction. The angle between first section 110 and second section 120 necessary to bridge the occipital-cervical junction may be characterized as a bend having a discrete angle or may be defined by an arc having a radius of curvature. In prior art approaches, this sharp bend in a support rod was shaped during the surgery process, with no time to heat-treat (anneal) the shaped rod.

In FIGS. 1A and 1B, cranial support rod 100 is shown without attaching hardware for illustrative purposes, but those skilled in the art will appreciate that attaching hardware is well known. Section 110 or section 120, or both, may be adapted for attachment to attaching hardware. For example, section 110 or 120 may be rounded or tapered on the end for easier insertion into a bone screw, the surface may be bead blasted, knurled, etched, or otherwise mechanically or chemically adapted for selected contact with attaching hardware. It would be further understood that section 110 can have a flattened profile to support the cranium or can attach to an occipital plate.

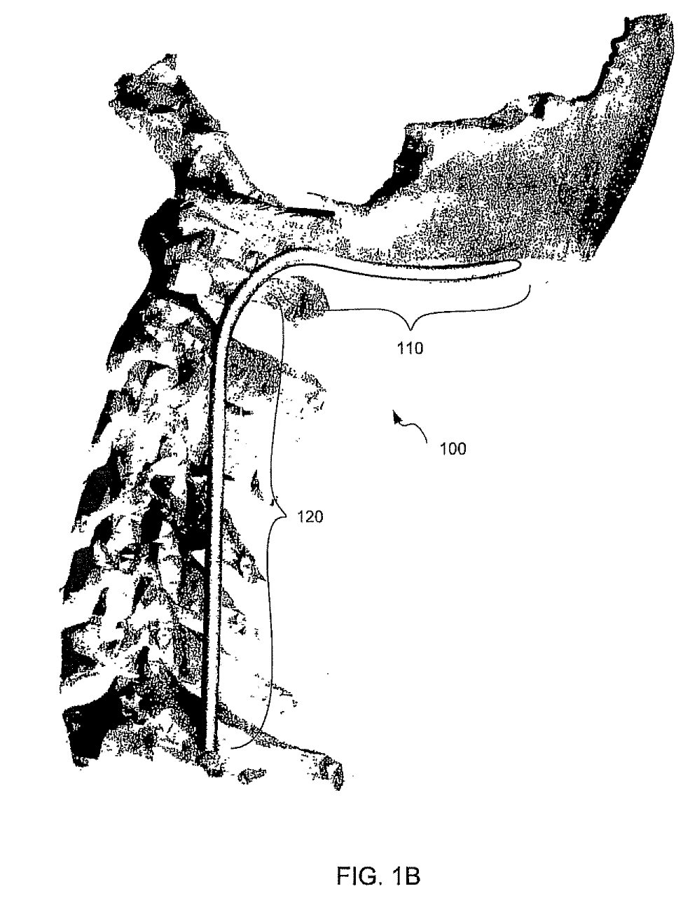

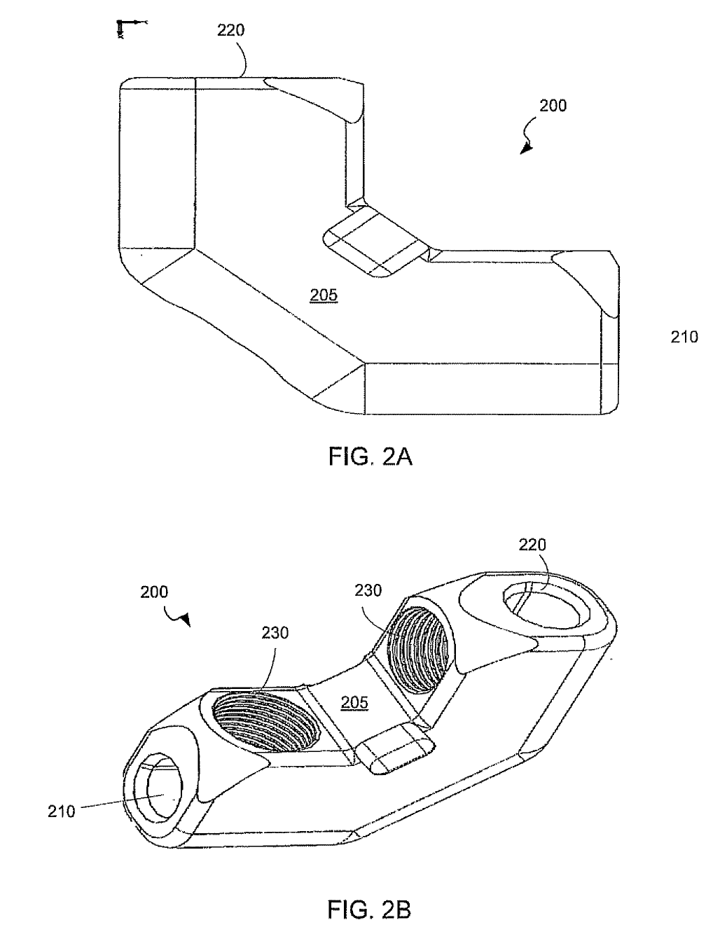

A cranial support rod 100 that is preconfigured to contour to the curvature of the occipital bone or a portion of the cervical spine and then stress relieved is advantageous over the prior devices for supporting the cranium In other cases, angled connectors can be used to connect straight rods or shaped rods to transition at the occipital-cervical junction. Embodiments of the present invention include angled connectors 200 and 300 as shown in FIGS. 2A-B, 3A-D, and 4. Angled connectors 200 and 300 are advantageous over prior art connectors and cranial support rods because angled connectors 200 and 300 transition the weight across sharp angles. Angled connectors 200 and 300 allow medical professionals to support more weight across sharper angles than using just a support rod that is shaped in surgery, but without the bulk associated with larger support mechanisms.

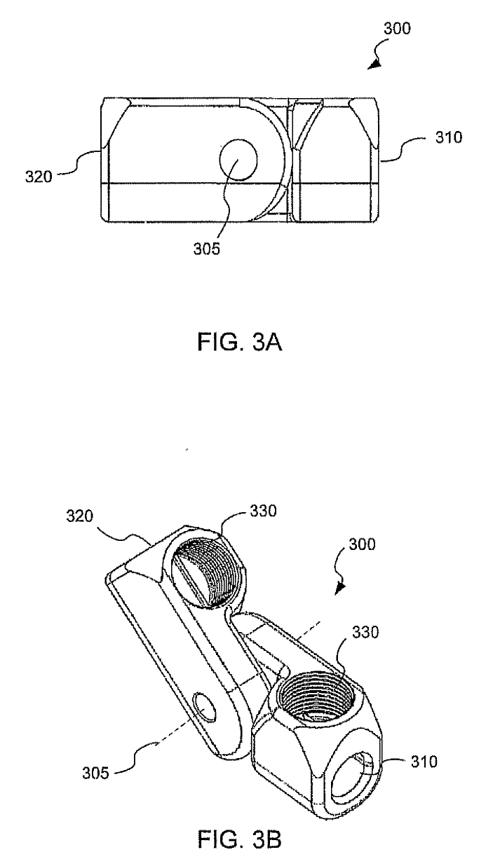

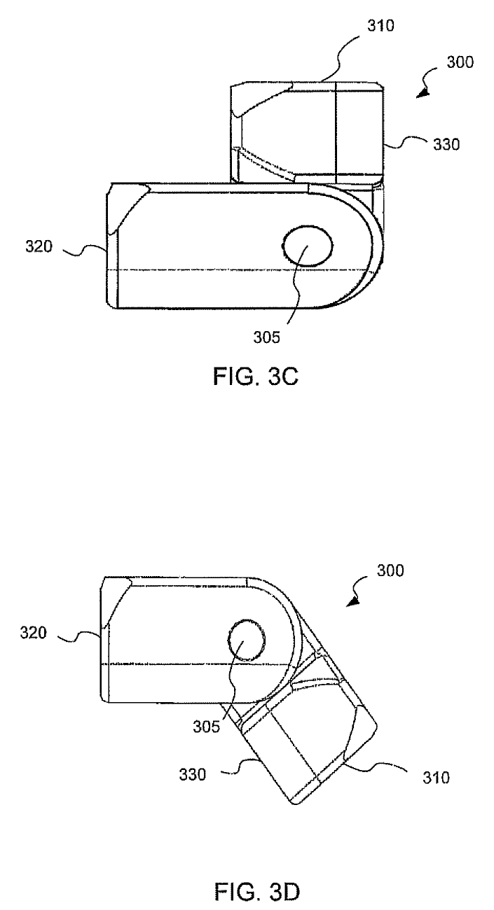

Angled connectors 200 or 300 may be pre-configured to a selected angle, as shown in FIGS. 2A-B, or may be adjustable such as by rotation before or during implantation, as shown in FIGS. 3A-D.

In the embodiment shown in FIGS. 2A-B, angled connector 200 includes a body 205 having a first receiver 210 and a second receiver 220, both of which are adapted, such as with opening 230 for a setscrew, for securely connecting to support rods. The terms secure and securely refer to a connection that may be temporary or permanent, but that will not disconnect without assistance from medical professionals. Cam-locks, taper-locks, and other adaptations may also be utilized to securely connect cranial support rods to angled connectors 200, 300 in different situations for different advantages without departing in scope from the present invention.

Angled connector 200 may be manufactured from any material that is biocompatible for implantation. Examples include, but are not limited to, stainless steel, commercially pure titanium, titanium alloys. In preferred embodiments, angled connector 200 is manufactured from commercially pure Grade 4 titanium to have the fatigue strength, biocompatibility, and other desirable characteristics. Angled connector 200 may be cast, machined, forged or any combination forming a single body 205 such that the necessary weight and stresses can be supported while minimizing the size of body 205. In other embodiments, receiver 210 and 220 are cast, machined, forged, or otherwise manufactured and then joined mechanically (such as riveted), chemically (such as epoxies), or thermally (such as welded) together to form body 205. Once manufactured, angled connector 200 may be treated for selected characteristics. Treatments may be for improved mechanical properties, such as corrosion resistance, or for improved biocompatibility, such as an infection control agent. Anodizing is one example of a treatment that may be applied to angled connector 200.

In the embodiments shown in FIGS. 2A-B, angled connector 200 is manufactured to have a fixed angle between first receiver 210 and second receiver 220. Although angled connector 200 is depicted in FIGS. 2A-B having a ninety degree angle, the present invention is not so limited, and embodiments of the present invention may be manufactured or configured to have any angle, such as forty five, sixty, ninety, one-hundred fifty, one hundred, eighty, two-hundred twenty five, and two hundred seventy degrees.

Angled connector 200 in FIGS. 2A and 2B is shown having first receiver 210 and second receiver 220 with circular profiles. However, embodiments of the present invention may be manufactured having receivers 210 and 220 with rectilinear, curvilinear, or other dimensions. First receiver 210 and second receiver 220 may have chamfered edges, tapered ends, or other machined adaptations for securely holding the angular support rods without creating stress risers/ shear forces, or other undesirable effects. Advantageously, angled connector 200 may be heat-treated, such as by annealing, to improve mechanical characteristics so that the size of angled connector 200 may be minimized.

In the embodiment shown in FIGS. 3A-D, angled connector 300 has a first receiver 310 adapted for adjustable connection (e.g. rotatably connected) about axis 305 to second receiver 320, both of which are adapted, such as with setscrew in threaded opening 230, a cam lock, or other mechanism, for securely receiving cranial support rods

First and second receivers 310 and 320 may be cast, machined, forged or a combination to provide the necessary strength and mechanical properties plus the ability to rotate relative to each other about axis 305.

First receiver 310 and second receiver 320 rotate about axis 305 to maintain cranial support rods in selected configurations. Rotation about axis 305 can be achieved by use of a rivet, bearing, or other mechanical element positioned between first receiver 310 and second receiver 320. Rotation about axis 305 may also be possible by incorporating mechanical elements in receivers 310 and 320. For example, receiver 310 may have a substantially cylindrical extension and receiver 320 may have a complementary circular cross-sectioned cavity or passage to receive the extension such that receiver 310 and 320 are rotatably connected while the extension rotates in the cavity or passage.

The position of receivers 310 and 320 about axis 305 are adjustable such that a medical professional may configure receivers 310 and 320 at a selected angle. For example, FIG. 3A depicts an angled connector configured to maintain two rods at an approximately one hundred eighty degree angle. FIG. 3B depicts an angled connector configured to maintain two rods at an approximately one hundred thirty five degree angle. FIG. 3C depicts an angled connector configured to maintain two rods at an approximately ninety degree angle. FIG. 3D depicts an angled connector configured to maintain two rods at an approximately two hundred twenty five degree angle.

In some embodiments, radial mating splines located on receiver 310 and receiver 320 surrounding axis 305 allow a medical professional to adjust the angle of angled connector 300 by rotating the first and second receivers 310 and 320 into a desired pre-selected configuration, then lock the receivers 310 and 320 to prevent further rotation. In other embodiments, connector 300 is adapted for adjustment such that receivers 310 and 320 are adjustable to any angle in a selected range of angles

In some embodiments, receivers 310 and 320 are adapted for selected rotation after implantation.

For example, mating splines on receivers 310 and 320 may allow rotation between two angles, such that the cranium is supported yet some movement is possible. Advantageously, allowing receivers 310 and 320 to move within a selected range of rotation to stabilize motion at the joint without diminishing support is desirable for comfort, fusion prevention, increased functionality, or other benefits. Such an angled connector 300 may also facilitate connections across joints, particularly sharp angled joints, without bending the cranial support rods Axis 305 may be operable to eliminate all movement between the range of angles, may have a frictioned surface to impede, but not eliminate, rotation of receivers 310, 320, or may incorporate machine elements such as cams or springs to provide dampened motion of receivers 310 and 320. In these embodiments, movement at the joint is stabilized to prevent injury, but rotatable to preserve motion. FIG. 3B depicts an angled connector configured to maintain two rods at an approximately one hundred thirty five degree angle, and further shows setscrews 330 which may be used to secure support rods into receivers 310 and 320.

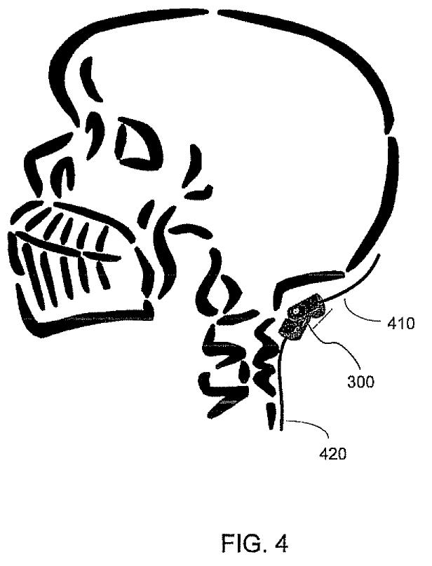

FIG. 4 shows one embodiment that uses angled connector 300 and two support rods 410 and 420 to support the cranium. In this embodiment, angled connector 300 maintains support rod 410 at an angle to support rod 420 such that the position of the cranium is maintained by bridging the occipital-cervical junction. Moreover, angled connector 300 supports the cranium across the sharp angle evident at the occipital-cervical junction.

Lower support rod 410 is configured to contour a portion of the cervical spine and may connect to a vertebra using bone screws or other attachment methods known in the art. Upper support rod 420 is similarly adapted to connect to the occipital bone and configured to contour to a portion of the occipital bone. Connection of lower support rod 410 to the cervical spine and upper support rod 420 to the occipital bone may be accomplished by affixing an anchor such as a bone screw in the bony tissue and attaching a support rod to the anchor using techniques known in the art. In some embodiments, lower support rod 410 and upper support rod 420 have a surface texture, coating or layer for selected attachment to a bone screw. For example, the surface or end of rod 410 or 420 may be bead-blasted, knurled, or otherwise machined to facilitate insertion into a receiver, for increased friction to prevent slippage once positioned, for improved biocompatibility or the like. The surface or end of rod 410 or 420 may be chemically etched, coated, anodized, or otherwise chemically treated for improved visibility in fluoroscopic images, for infection resistance, or the like.

Angled connector 300 is adapted to securely maintain support rods 410 and 420 at a selected angle relative each other and further adapted to transition the force of the cranial weight from support rod 410 to support rod 420 across the sharp angle of the occipital-cervical junction. Advantageously, angled connector 200 may be selected for a desired angle, or angled connector 300 may be adjusted to a desired angle such that the sharp angle of the occipital-cervical junction is bridged by angled connector 200 or 300 and therefore cranial support rods 410 and 420 require minimal bending—both in the degrees bent and the number of bends—to connect to angled connector 300. In doing so, the strength and other mechanical properties of cranial support rods 410 and 420 are preserved.

Contoured rods that have been shaped to contour to the occipital bone or to a portion of the cervical spine and then annealed or otherwise treated to reduce stress advantageously require less bending, twisting, and other manipulation during surgery that is detrimental to mechanical properties (e.g., fatigue strength, tensile strength and shear strength), and reduces the time in surgery because less manipulation is needed In contrast, prior art rods (particularly ones that are not pre-bent or are not pre-shaped to configure to either the occipital bone or the cervical spine) require some manipulation, which requires time and which changes the mechanical properties of the rods. Advantageously, the rods 410 and 420 may still be shaped at the time of surgery with less detriment to the integrity of the rods. Although bending, twisting, or other manipulations may impact the desired characteristics (e.g. fatigue strength, shear strength, torsional strength, stiffness, and/or hardness) of rod 410 or 420, the pre-shaped rods 410 and 420 would need less manipulation than prior art approaches. Furthermore, although manipulation may increase the internal stresses in rods 410 and 420, they do not experience the same stress as in prior art methods because the bend at the occipital-cervical junction is handled by angled connector 300. Fatigue strength and shear strength, which may have the most impact on the success or failure of any cranial support system, are preserved by providing an angled connector to bridge the sharp angle, along with using shaped, heat-treated rods that contour to the occipital bone and cervical spine.

In the foregoing specification, the invention has been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Benefits, other advantages, and solutions to problems have been described above with regard to specific embodiments However, the benefits, advantages, solutions to problems, and any components) that may cause any benefit, advantage, or solution to occur or become more pronounced are not to be construed as a critical, required, or essential feature or component of any or all the claims.

What is claimed is:

1. An angled support rod for stabilizing movement of a cranium comprising:
a first section shaped to contour to the curvature of an occipital bone; and
a second section shaped to contour to the curvature of a portion of the cervical spine;
wherein the first section and second section form an angle configured for bridging the occipital-cervical junction of a body, and the support rod is heat-treated.

2. The cranial support rod of claim 1, wherein the rod is annealed

3. The angled support rod of claim 1, wherein the angle formed between the first section and the second section comprises a curve having a selected radius.

4. The cranial support rod of claim 1, wherein the rod cross-sectional dimension is uniform along its length.

5. A method for manufacturing a cranial support rod, comprising:
shaping a first section of a rod to contour the curvature of a portion of the occipital bone;
shaping a second section of the rod to contour to the curvature of a portion of the cervical spine;
configuring the rod to bridge the occipital-cervical joint; and
heat treating the cranial support rod to reduce stress.

6. The method of claim 5, wherein heat treating comprises annealing.

7. The method of claim 5, wherein the rod is configured to form a selected angle between the first section and the second section.

8. An angled connector for maintaining the cranium at a selected angle to the cervical spine comprising:
a first receiver adapted for secure connection to a first support rod attached to an occipital bone; and
a second receiver connected to the first receiver and further adapted for secure connection to a second support rod attached to a portion of the cervical spine,
wherein the connection between the first receiver and second receiver maintains an angle between the first and second support rods.

9. The angled connector of claim 87 wherein the angle is between thirty and one-hundred eighty degrees.

10. The angled connector of claim 8, wherein the connection between the first and second receivers comprises a rotatable connection.

11. The angled connector of claim 10, wherein the rotatable connection is configurable to pre-set angles, wherein the angle between the first and second directions is one of the pre-set angles.

12. The angled connector of claim 8, wherein the adaptation comprises a setscrew.

13. A system for supporting the cranium, comprising:
an angled connector comprising
a first receiver adapted for secure connection to a first support rod extending in a first direction from the first receiver; and
a second receiver connected to the first receiver and further adapted for secure connection to a second support rod extending in a second direction from the second receiver,
wherein the first receiver and second receiver are connected to maintain an angle between the first and second directions, wherein the angle is selected for bridging the occipital-cervical junction;
a first support rod configured for insertable connection to the first receiver, wherein the first support rod is shaped to contour to an occipital bone and heat-treated to a low-stress state; and
a second support rod configured for insertable connection to the second receiver, wherein the second support rod is shaped to contour to a portion of the cervical spine and heat-treated to a low-stress state.

14. The system of claim 13, wherein the angle between the first and second receivers is between thirty and one-hundred eighty degrees.

15. The system of claim 13, wherein the connection between the first receiver and the second receiver is adjustable.

16. A method of supporting a cranium comprising:
attaching a first section of a heat-treated cranial support rod to a portion of the occipital bone, wherein the first section is shaped to contour to the curvature of the occipital bone; and
attaching a second section of the cranial support rod to a portion of the cervical spine, wherein the second section is shaped to contour to the curvature of a portion of the cervical spine,
wherein the rod is configured to maintain an angle between the first section and the second section based on the occipital-cervical junction,

17. A method for supporting the cranium comprising:
connecting a first support rod to an angled connector,
wherein the angled connector comprises
a first receiver adapted for secure connection to a first support rod extending in a first direction from the first receiver; and
a second receiver connected to the first receiver and further adapted for secure connection to a second support rod extending in a second direction from the second receiver,
wherein the first receiver and second receiver are connected to maintain an angle between the first and second directions;
connecting a second support rod to the angled connector;
connecting the first support rod to a portion of the occipital bone, wherein the first support rod is shaped to contour to the occipital bone and further heat-treated to a low-stress state; and
connecting the second support rod to a portion of the cervical spine wherein the second support rod is shaped to contour to a portion of the cervical spine and further heat-treated to a low-stress state.

18. The method of claim 17, wherein the angled connector comprises:
a first receiver adapted for secure connection to the first support rod extending in a first direction from the first receiver; and
a second receiver connected to the first receiver and further adapted for secure connection to the second support rod extending in a second direction from the second receiver,
wherein the first receiver and second receiver are connected to maintain an angle between the first and second directions.

19. The angled connector of claim 30, wherein the angle is between thirty and one-hundred eighty degrees.

20. The angled connector of claim 30, wherein the connection between the first and second receivers comprises a rotatable connection.
